(19) United States
(12) Patent Application Publication
(10) Pub. No.: US 2010/0159579 A1
(43) Pub. Date: Jun. 24, 2010

(54) PHOTOBIOREACTOR SYSTEMS

(76) Inventors: Christopher S. Schuring, Penryn, CA (US); J. Kyle McCue, San Jose, CA (US)

Correspondence Address: WILSON, SONSINI, GOODRICH & ROSATI, 650 PAGE MILL ROAD, PALO ALTO, CA, 94304-1050, US

(21) Appl. No.: 12/582,697

(22) Filed: Oct. 20, 2009

Related U.S. Application Data
(60) Provisional application No. 61/106,962, filed on Oct. 20, 2008.

(51) Int. Cl. C12M 1/00

(52) U.S. Cl. 435/292.1

(57) ABSTRACT

The invention provides for photobioreactor systems that can be used for the growth of photoautotrophic organisms. The photobioreactor systems can be scalable and modular, such that the production capacity of a photobioreactor system can be readily increased or decreased. The system may include photobioreactor units or blades that can be operated and maintained through a central control system.

### CROSS-REFERENCE

This application claims the benefit of U.S. Provisional Application No. 61/106,962, filed Oct. 20, 2008, which application is incorporated herein by reference.

### BACKGROUND OF THE INVENTION

The commercial potential of producing biomass products by photosynthesis techniques using simple plant matter, such as algae, blue green bacteria, and seaweed, has been recognized. Such techniques seek to harness the ability of photoautotrophic organisms to utilize sunlight and carbon dioxide to produce biomass products.

Methods involving open-systems for cultivation of photoautotrophic organisms have been attempted. However, such methods have been impractical for numerous reasons, including contamination, low yield, loss of water, and inefficient use of light.

Closed-system photobioreactors have been designed to address these limitations. Examples of such systems have been described in GB Patent No. 2,118,572, U.S. Pat. No. 7,176,024, PCT Publication No. WO 94/09112, PCT Publication No. WO2005/059087, PCT Publication No. WO 2007/070452, and U.S. Pat. No. 5,242,827, each hereby incorporated by reference. However, these systems are not readily increased in scale and are not space-efficient. Therefore, there is a need for a photobioreactor system that addresses these limitations.

### SUMMARY OF THE INVENTION

The invention provides for photobioreactor systems that can be used for growth of photoautotrophic organisms. The photobioreactor systems can be scalable and modular, such that the production capacity of a photobioreactor system can be readily increased or decreased.

The photobioreactor systems described herein can include hives, clusters, and pods. A pod can have multiple blades connected to a backplane, where the joining of a blade to a backplane creates a functional photobioreactor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the invention may be further explained by reference to the following detailed description and accompanying drawings that sets forth illustrative embodiments.

FIG. 1 shows a diagram of a photobioreactor hive made up of three clusters, each cluster having three pods, and each pod having six blades.

FIG. 2 shows a diagram of a cluster made up of three pods.

FIG. 3 shows a diagram of a pod.

FIG. 4 shows a schematic of a blade having serpentine tubes and multiple sensors.

FIG. 5 shows an end-on-view of a rail.

FIG. 6 shows a side-view of a rail.

FIG. 7 shows a schematic of a clevis hanger.

FIG. 8 shows an end-on-view of a tube.

FIG. 9 shows an end-on-view of a tube.

FIG. 10 shows a front-view and a side-view of a backplane.

FIG. 11 shows a top-view of a backplane.

FIG. 12 shows a front-view of a backplane with multiple tanks.

FIG. 13 shows a side-view of a backplane with multiple pumps.

FIG. 14 shows a side-view of a blade connected to a backplane.

FIG. 15 shows a view of a pod.

FIG. 16 shows a top-view of a pod.

FIG. 17 shows a front-view of a pod.

FIG. 18 shows a side-view of a pod.

FIG. 19 shows a back-view of a pod.

FIG. 20 shows a schematic of a photobioreactor system.

FIG. 21 shows an exploded view of a rack.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

Various aspects of the invention provide for photobioreactor systems that can be utilized for growth of microorganisms, such as photoautotrophic organisms. The photoautotrophic organisms grown in the photobioreactor systems can be utilized for sequestering and/or recycling carbon dioxide and/or for producing of biomass. The biomass can be, for example, algae, a biofuel, an animal feed, a pharmaceutical, or a nutraceutical (e.g. astaxanthin). Preferably, the photobioreactor systems are scalable systems that can be configured to the needs of a particular site. The scalable photobioreactor systems can have increased or decreased capacity by the addition or removal of modules. The photobioreactor systems can be designed to be space-saving, allowing for increased productivity per area.

The photobioreactor systems disclosed herein can be closed-loop, self-contained systems. This can reduce the effects of weather changes and reduce the chance of contamination by pollution, rogue algae species, or wind-borne contaminants.

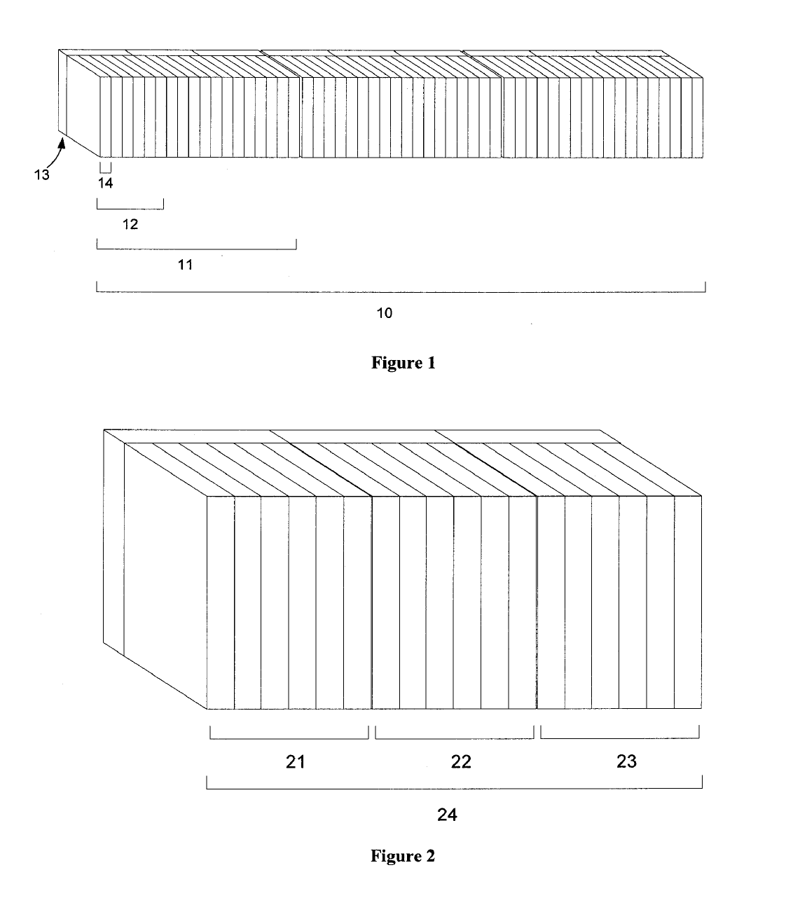

An example of a scalable and modular photobioreactor system is shown in FIG. 1. The photobioreactor system (10), herein also called a hive, may include three clusters or blocks (11) and nine pods (12). Each pod may have one backplane (13) and six blades (14) for a total of nine backplanes (13), and fifty four blades (14). The capacity for growth of photoautotrophic organisms, for sequestering carbon dioxide and/or producing of biomass, can be scaled by the addition or removal of a module such as a blade, a pod, a cluster, or a hive. A hive, cluster, or pod can have any number of modules. For example, a hive can have one, two, three, four, or more clusters. Within a pod, a blade can have a reservoir for growth of a photoautotrophic organism and a backplane can have equipment such as pumps and electrical controls that interface with one or more blades.

Within a hive, one or more clusters can share resources by fluid and electrical connections. Each cluster can have a fluid connection to a central unit and/or can have a fluid connection to another cluster so as to have a parallel and/or serial arrangement of clusters. The central unit can provide a variety of functions, for example, the central unit can be a harvesting unit for recovery of biomass. The fluid connections can be used to provide water, nutrients, and/or photoautotrophic organisms to the clusters or for sharing water, nutrients and/or photoautotrophic organisms among the clusters. Similar to the fluid connections, clusters can have electrical connections that are arranged in a parallel and/or serial configuration. The electrical connections can be used to supply power and/or for the communication of signals between photobioreactor components or between photobioreactors and a central unit. The central unit may be a central processing unit. A clusters or hive can be operated independently, or in conjunction with another cluster and/or hive.

FIG. 2 shows an example of a cluster (24) having three pods (21, 22, 23). The three pods can have fluidic and electrical connections for sharing resources. Alternatively, the three pods can be operated independently of each other. The fluidic and electrical connections between the pods can be serial and/or parallel connections. A pod can be operated independently, or in conjunction with another pod.

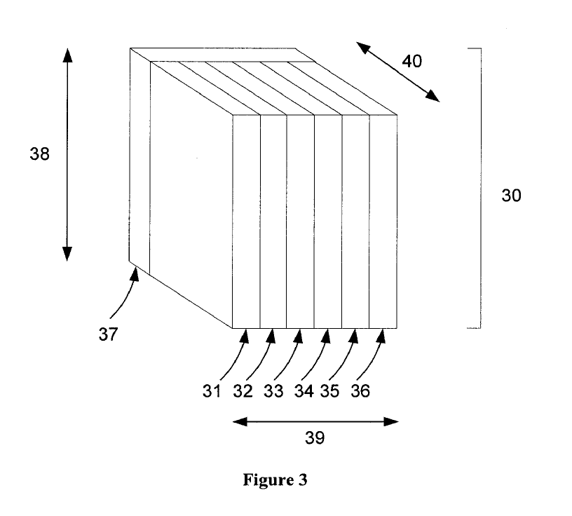

FIG. 3 shows a pod (30) having a backplane (37), and six blades (31, 32, 33, 34, 35, 36). A pod can have any number of blades, depending on the design of the system. A blade can have a reservoir for growing a photoautotrophic organism. A blade can have fluidic and electrical connections for transferring a fluid medium, powering the blade, and/or communicating signals. A blade can be operated independently, or in conjunction with another blade. The blade can be rackable in a frame, e.g. a frame of a pod. The reservoir can be a liquid-holding reservoir configured to expose one or more photoautotrophic organisms growing in the reservoir to light. Light supplied to the photoautotrophic organisms can be sunlight or artificial light. Supply of solar light can be aided by solar tubes and mirrors. The artificial light can be supplied by any light source known to those skilled in the art, such as a light emitting diode, a compact fluorescent light, or a grow light. The backplane can have one or more pumps, tanks, and electrical controls that interface with the one or more blades of a pod. The electrical controls of a backplane can interface with one or more sensors of a blade. The electrical controls can monitor the growth of a photoautotrophic organism and allow for control of environmental conditions within the photobioreactor system. The capacity of a pod for growth of a photoautotrophic organism or production of biomass can be increased or decreased by altering the number of blades per pod, or altering the dimensions of the pod. The pod can have a height (38), width (39) and depth (40). Increasing the height, width, and/or depth can increase the capacity of the pod for growth of photoautotrophic organism or production of biomass.

Use of a blade and backplane system for forming photobioreactors allows for isolation of photoautotrophic organism cultures. This can allow for reduced chance of contamination and improved optimization of productivity. For example, under-producing cultures can be eliminated while high-producing cultures can be selected for subsequent rounds of growth. Additionally, the blade and backplane system can allow for grouping of similar mechanical and electrical components. All tanks, pumps, and electrical controls can be placed on a backplane and maintained separately from a liquid-holding reservoir for exposing photoautotrophic organisms growing within the photobioreactor to light. Separation of components can allow for components with similar life expectancies to be grouped, which can reduce maintenance cost of the photobioreactor system.

A photobioreactor system may include a blade connected to a backplane. The joining of a blade to a backplane can be a functioning photobioreactor. The blade can have a plurality of horizontal tubes that are in end-to-end fluid connection with each other and can form a liquid-holding reservoir. The tubes can be connected end-to-end using elbow connections. The horizontal, or substantially horizontal, tubes can be arranged or stacked vertically to save space. Alternatively, the tubes can be aligned vertically and the arrangement of tubes can be in a horizontal direction. The number or size of tubes can be increased or decreased to change the volumetric capacity of the blade. In some embodiments of the invention, a blade's height can be increased to increase volumetric capacity of a blade while not altering the footprint of the blade. The tubes can be optically transparent to allow transmission of light through the tubes. Alternatively, the tube can be configured to not allow the transmission of light through the tubes, as described herein. The tubes can be supported between two plates, or any other means known to those skilled in the art. The configuration of the tubes can be optimized for distribution of light, volumetric capacity per area of land used, for optimal growth of a photoautotrophic organism, and/or for optimal production of a biomass product.

A blade and backplane system can be self-cleaning. Examples of cleaning systems are described in PCT Publication No. WO94/09112, U.S. Pat. No. 5,242,827, and U.S. Pat. No. 6,370,815, each hereby incorporated by reference.

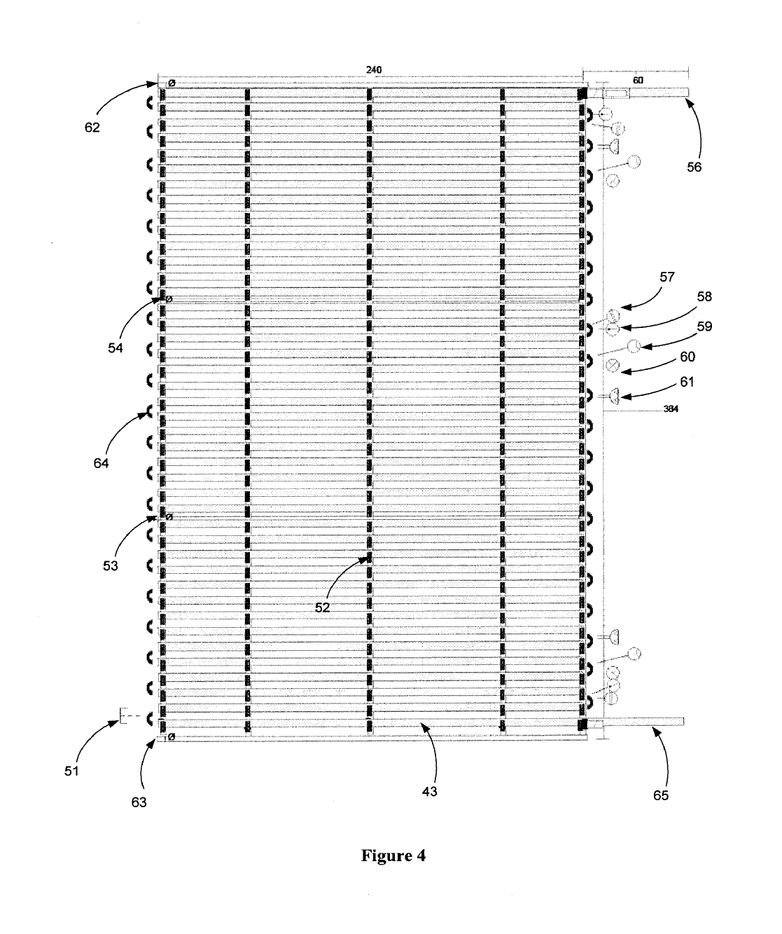

FIG. 4 shows an embodiment of a BioBlade™ having a plurality of horizontal tubes (43) that are in fluid connection. The blade can be utilized for growth of a photoautotrophic organism. The tubes can be of any dimension. In some embodiments of the invention, the tubes are four inch clear PVC pipes that are 10 feet in length. In other embodiments of the invention, the tubes are borosilicate tubes that are transparent to light. The tubes described herein can be coated with a reflective material to increase the amount of light that can be directed to a photoautotrophic organism. Additionally, the reflective material can improve thermal management of the photobioreactor system. Alternatively, the tubes can be configured to not allow for transmission of light through walls of the tube. For example, the tubes can be coated with a material that is 100% light reflective, or the tubes can be constructed of an material that is not light transparent. The tubes can be joined by elbow joints (64) and, collectively, can form a liquid-holding reservoir. The reservoir can have an inlet (65) and an outlet (56). A liquid medium, for example a growth medium, can be pumped into the inlet, passed through the plurality of tubes, and exit through the outlet. Alternatively, the liquid medium may be pumped in the opposite direction. The inlet and outlet of the blade can be designed for fluid connection to a backplane. The fluid connection between the blade and the backplane can be any type of connection, for example, a quick-release with an automatic closure feature upon disconnection, or a screw connection. A manual drain valve (51) can be positioned near the bottom of the liquid holding reservoir for draining. The backplane and blade can also have connection for communicating signal between any of the plurality of sensors on the blade to the backplane. The connection for communicating signal can be wired or wireless. In some embodiments of the invention, one or more wired electrical connections between the blade and the backplane can allow for transmission of power and electrical signals. The wired electrical connections can be joined by plugs, contact plates, or any other electrical connections known to one skilled in the art.

The tubes can be suspended by a rigid structure. The rigid structure can have a plurality of rails (63, 53, 54, 62) that support the plurality of tubes. Each tube can be connected to another tube or to a rail by a clevis hanger (52). FIG. 4 shows that each tube is supported by five clevis hangers; however, any number clevis hangers can be used per tube.

A blade can also have a plurality of sensors (57, 58, 59, 60, 61). The sensors can be utilized to measure density (57), temperature (58), flow rate (59), pressure (60), and pH (61). Additionally, sensors may measure light intensity, the concentration of a biomass product, or the concentration of a gas such as oxygen, carbon dioxide, or nitrogen. The measurements can be used to monitor the growth of a photoautotrophic organism or to monitor the production of biomass.

Sensors can be placed in multiple locations on a blade. For example, sensors can be placed near the top, middle, and bottom of the plurality of tubes, as shown in FIG. 4. Additionally, sensors can be placed on the blade chassis or the backplane. These sensors can be used to monitor environmental conditions, for example light intensity or temperature.

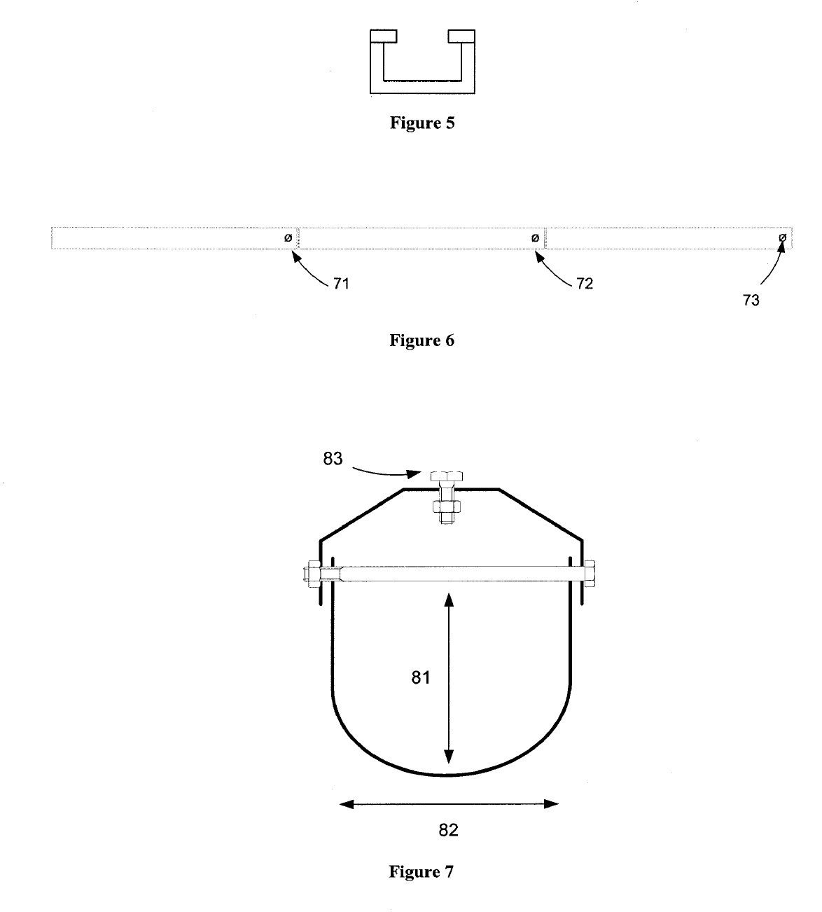

The rails of the rigid structure for supporting the plurality of tubes in a blade can be made of metal, glass, plastic, or any other material known to those skilled in the art. An end-on-view of a rail is shown in FIG. 5. A side-view of a rail assembly, having three rails, is shown in FIG. 6. As shown in FIG. 6, the rails can be connected to another rail at a connection point (71, 72, 73). This can allow for simplified transportation and assembly of a rigid structure. In some embodiments of the invention, a rail can be made of 14 gage steel with holes centered every two inches. The length of a rail assembly can be 10 feet long. The bottom and/or top rail of a rigid structure can have a set of rollers to facilitate insertion and removal of a blade into a pod. In some embodiments of the invention, the blades can be slid into and out of a chassis structure.

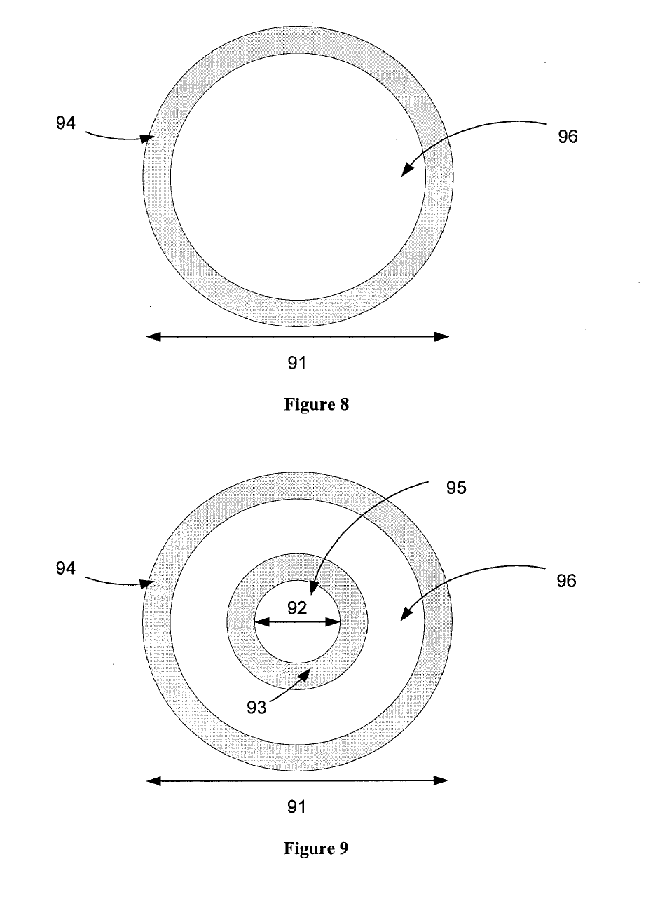

FIG. 7 shows a clevis hanger that can be used for attaching a tube to a rail. The clevis hanger can have a bolt (83) that can be secured to a rail. The clevis hanger can have an inside vertical dimension (81) of 5 inches and an inside horizontal dimension (82) of 4 inches.

FIG. 8 shows a preferable embodiment of tubes that can be used to form a liquid-holding reservoir of a blade. The tube (94) can enclose a space (96) for holding a fluid medium. The tube can have an outer diameter (91). The outer diameter can be any length, for example 1, 2, 3, 4, 5, 6, 7, 8, 9, or 10 inches.

FIG. 9 shows an exemplary embodiment of tubes that can be used to form a liquid-holding reservoir of a blade. In some embodiments of the invention, the liquid-holding reservoir can have an interior tube (93) located inside an exterior tube (94). This can create two spaces for a fluid medium. A first space (96) between the interior tube (93) and the exterior tube (94) and a second space (95) inside the interior tube (93). The interior tube (93) can be used for thermal regulation of a fluid medium contained within the tube (94). The fluid medium for thermal regulation can be ethylene glycol, oil, water, any combination thereof, or any other fluid medium known to those skilled in the art. The outside diameter of the exterior tube can be 4 inches. The ratio of the first space to the second space can be configured such that the temperature of a first fluid medium contained within the first space can be regulated by controlling the temperature and flow rate of a second medium contained within the second space. In some embodiments of the invention, both the interior and exterior tubes are transparent PVC tube. In other embodiments of the invention, the exterior and interior tubes can be made of any material such as metal, plastic, or glass, and need not necessarily be made of the same material.

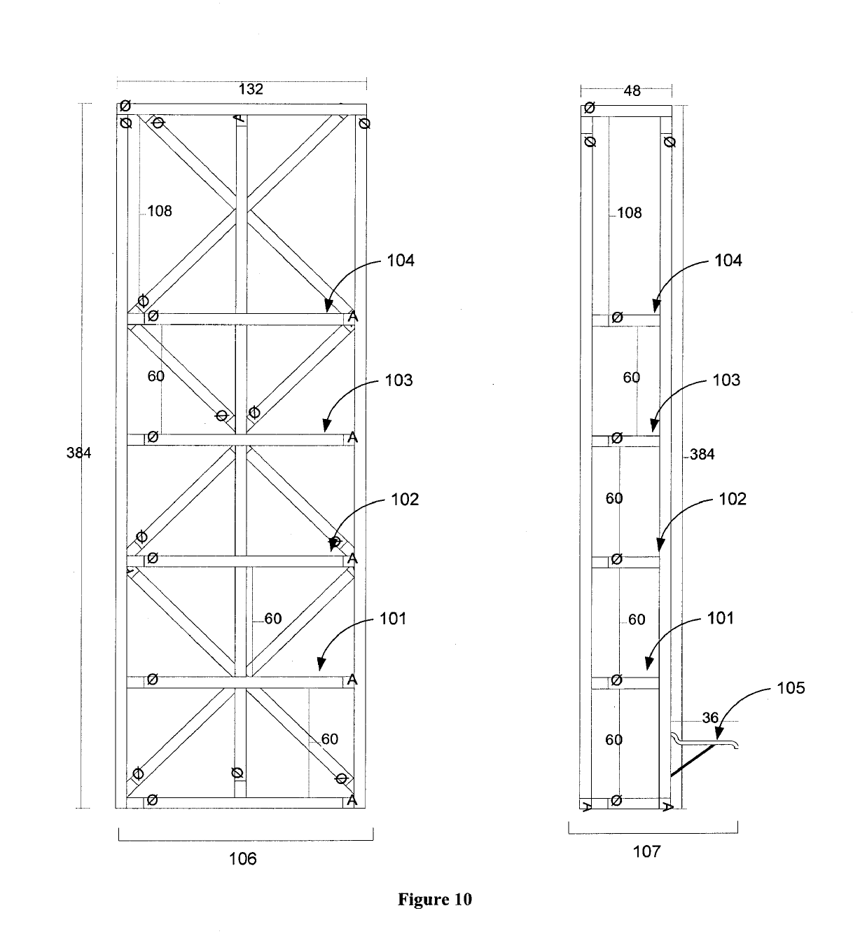

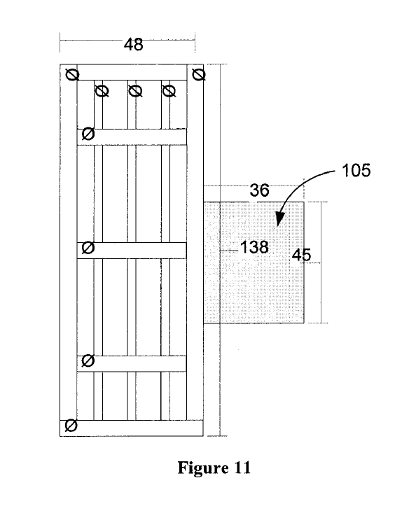

A backplane can have a backplane skeleton for supporting one or more photobioreactor components. FIG. 10 shows a front-view (106) and a side-view (107) of a BioPlane™ skeleton that can form part of a BioPod™. The backplane skeleton can be made of multiple rails that are connected to form a rigid structure. The rails can be the same type used to support tubes of a blade. The backplane skeleton can have one or more platforms (101, 102, 103, 104) for supporting the photobioreactor components. Additionally, the backplane skeleton can have a work platform (105). The work platform can facilitate user access to the photobioreactor components. The work platform can facilitate access to photobioreactor components by creating an elevated standing area so that a user can reach the photobioreactor components. FIG. 11 shows a top view of a backplane skeleton and the work platform (105) that facilitates access to the photobioreactor components.

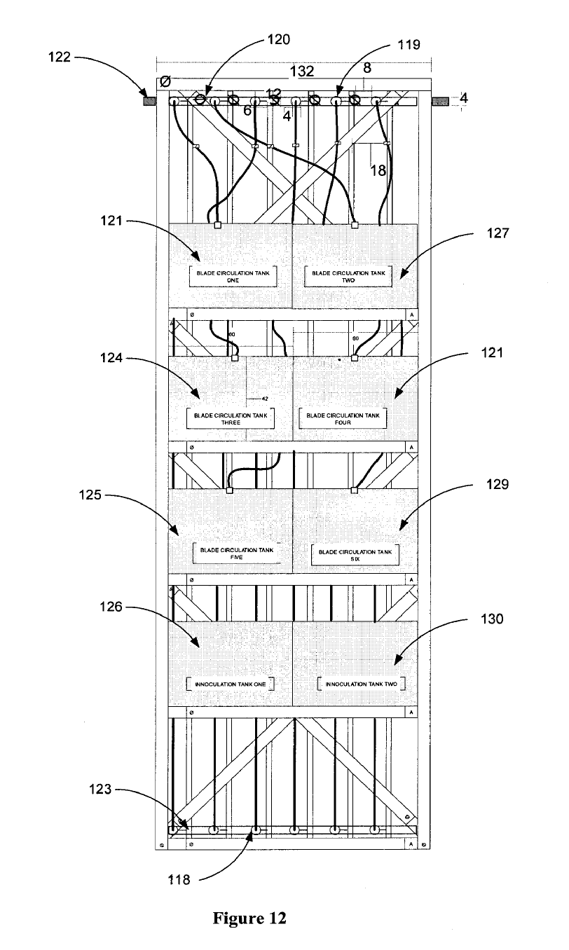

The photobioreactor components that are supported by the backplane can include one or more tanks, tubes that provide fluid connection between the components and the blades, pumps, electrical hardware, and electronic controls. FIG. 12 shows a front-view of a backplane having eight tanks (121, 124, 125, 126, 127, 128, 129, 130). These tanks can include circulation tanks and inoculation tanks. A circulation tank can be used to as a liquid-holding reservoir that does not expose a photoautotrophic organism contained within the liquid-holding reservoir to light. Alternatively, the circulation tank allows for improved mixing of a culture of photoautotrophic organisms. The inoculation tank can be used to store a photoautotrophic organism that can be used to inoculate a blade. Alternatively, the inoculation tank can be used as an initial liquid-holding reservoir for the initial growth stages of a photoautotrophic organism. For example, a growth medium not containing a photoautotrophic organism can be prepared in the inoculation tank, and then a photoautotrophic organism can be introduced to the inoculation tank, which can be performed by any methods known in the art. The photoautotrophic organism can be cultured within the inoculation tank until the culture reaches an intended density. Once the photoautotrophic organism has reached an intended density, the growth medium containing the photoautotrophic organism can be introduced to a blade.

In some embodiments of the invention, a blade can have a corresponding circulation tank. Each circulation tank can be connected to a single blade, or can be connected to manifolds (120, 123) at the top and bottom of the backplane by fluidic connections. In some embodiments of the invention, a tank can be connected to a blade using one or more junctions (119, 118). Each junction can control flow between a manifold (120, 123), a tank, and a blade. A junction, which can have a gate valve, can control the flow rate of a fluid medium between any two components. The manifold can be used to supply additional water or nutrients, such as carbon dioxide, to a blade. Additionally, an inoculation tank can be connected to a manifold, such the contents of an inoculation tank can be introduced to a blade. The fluidic connections between the tanks, the pumps, the blades, and the manifold can be rigid or flexible tubes. The backplane can also have in-line ports (122) for connection to another backplane.

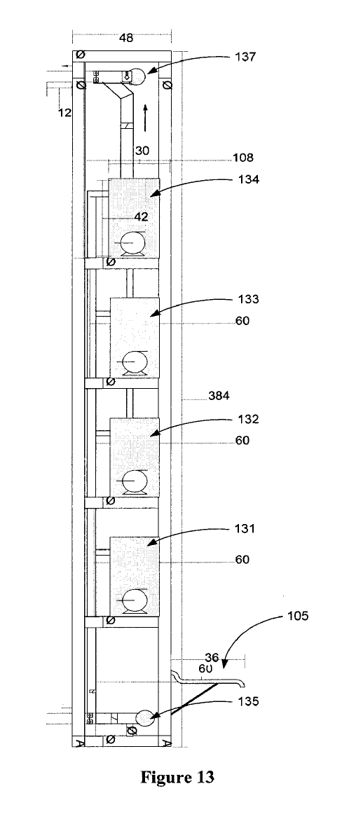

FIG. 13 shows a side-view of a backplane with a plurality of pumps (131, 132, 133, 134). The pumps can be used to circulate a fluid medium in the circulation tanks and throughout a blade. A pump can correspond to each tank that is supported by the backplane. The pumps can be diaphragm pumps, centrifugal pumps, peristaltic pumps, or any other pump known to those skilled in the art. Alternatively, fluid mediums can be moved through the photobioreactor systems using devices and methods other than pumps, such as bubbling of a gas, vacuum sources, thermal convection, and gravity. FIG. 13 also shows the connection points (137, 135) for connecting the photobioreactor components of the backplane to a blade. The bottom connection point (135) can have a harvest and drain line.

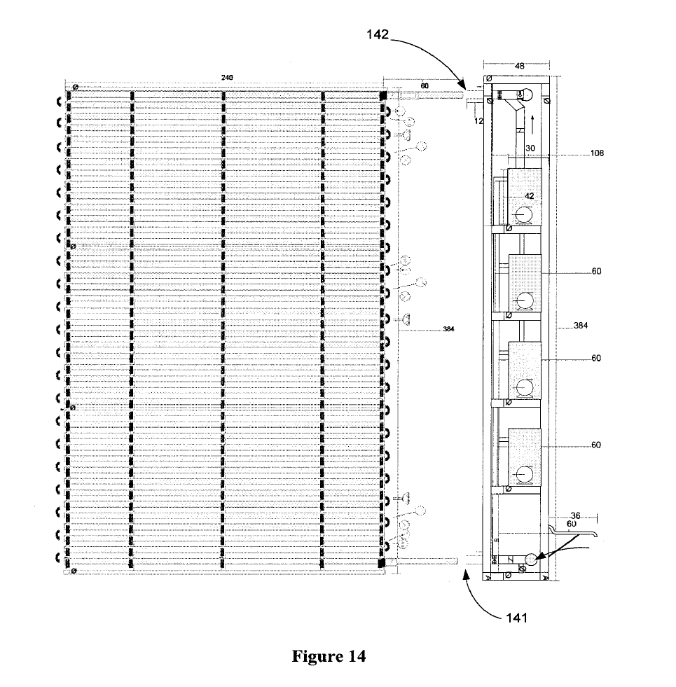

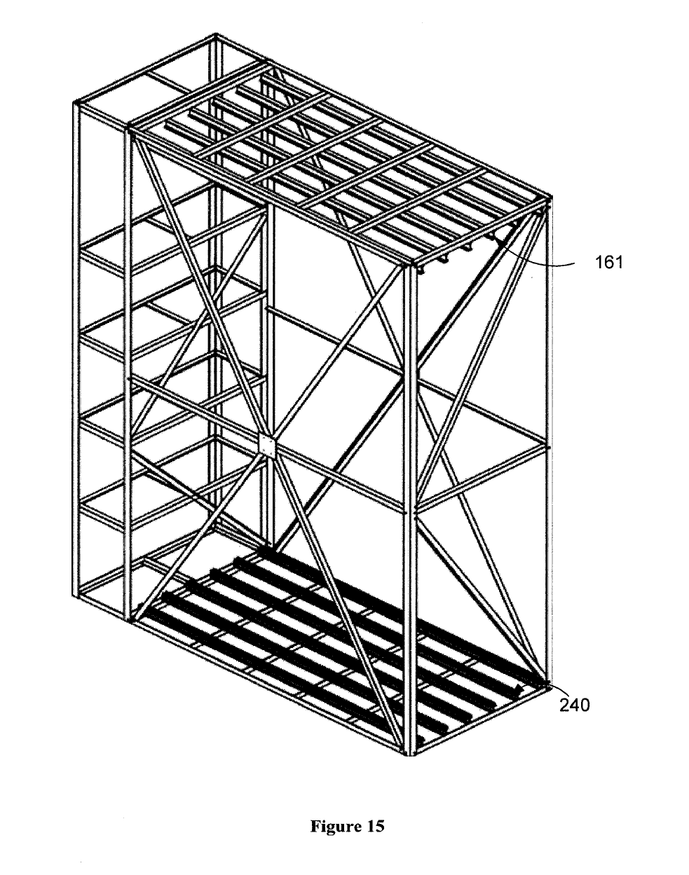

FIG. 14 shows a side-view of a blade aligned for connection to a backplane. The connection between the two components can occur at two locations (142, 141). The connections between the blade and the backplane can be at more than two locations. For example, the blade and the backplane can have two additional fluid connections about midway through the plurality of tubes. These additional fluid connections can be used to place an additional pump midway through the plurality of tubes, so as to reduce the amount of power required by a single pump to move a fluid medium through the photobioreactor system.

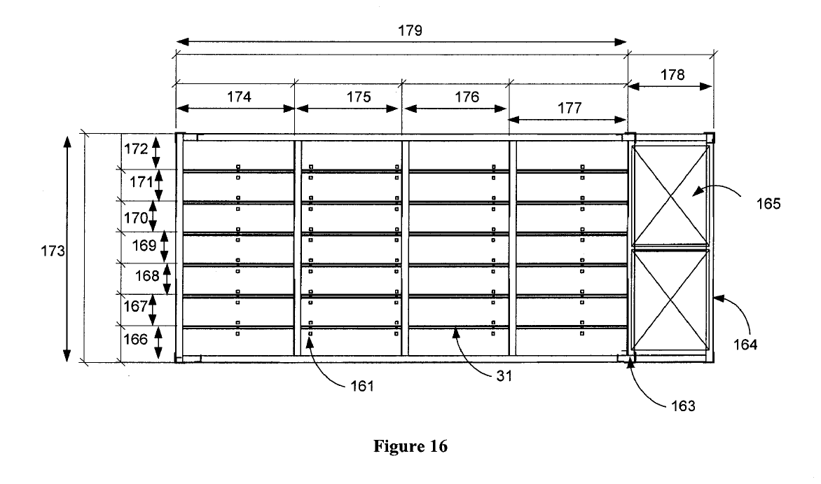

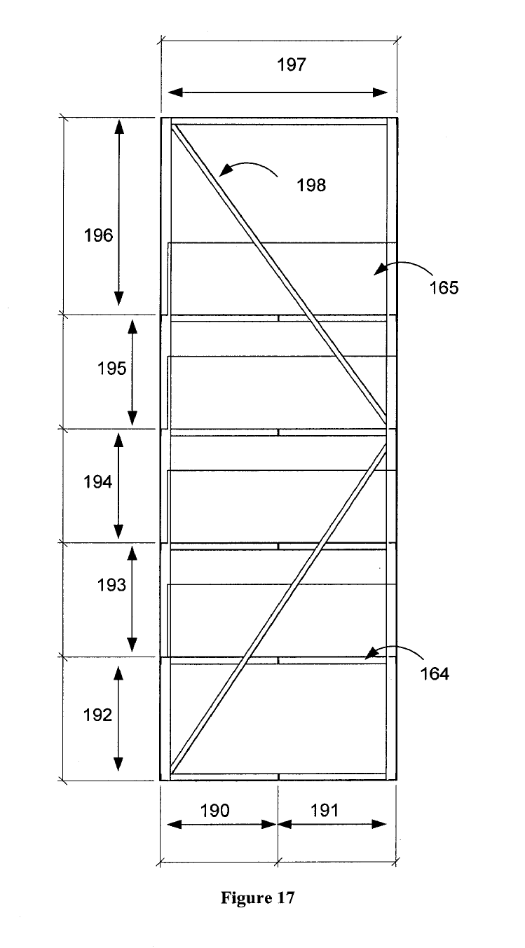

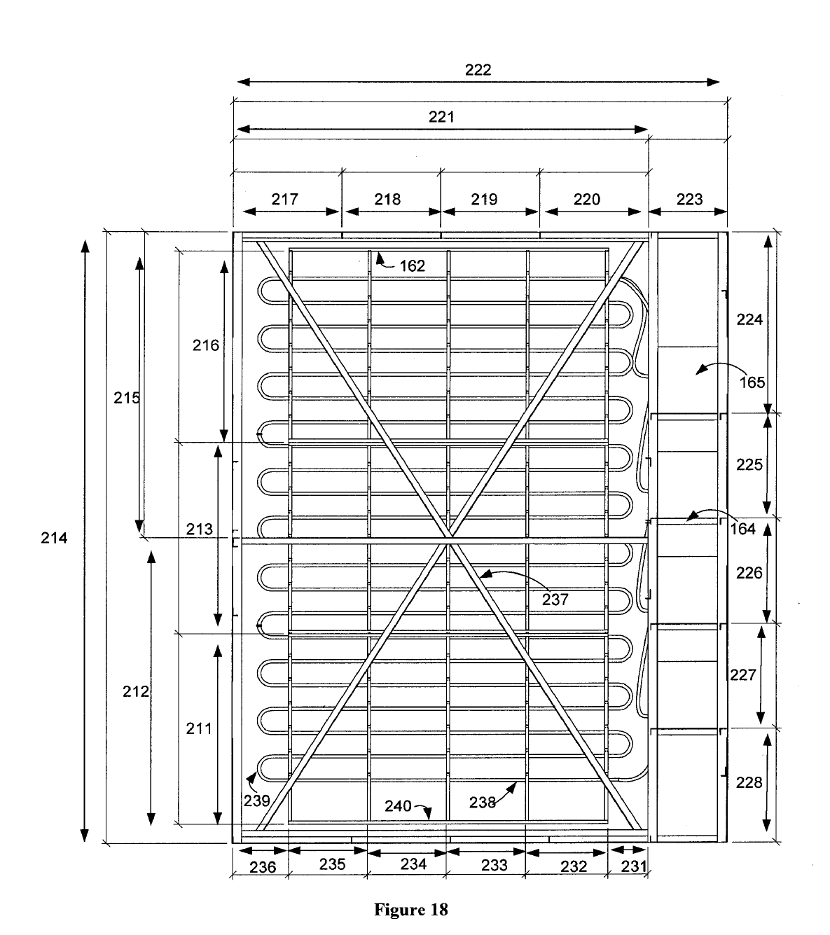

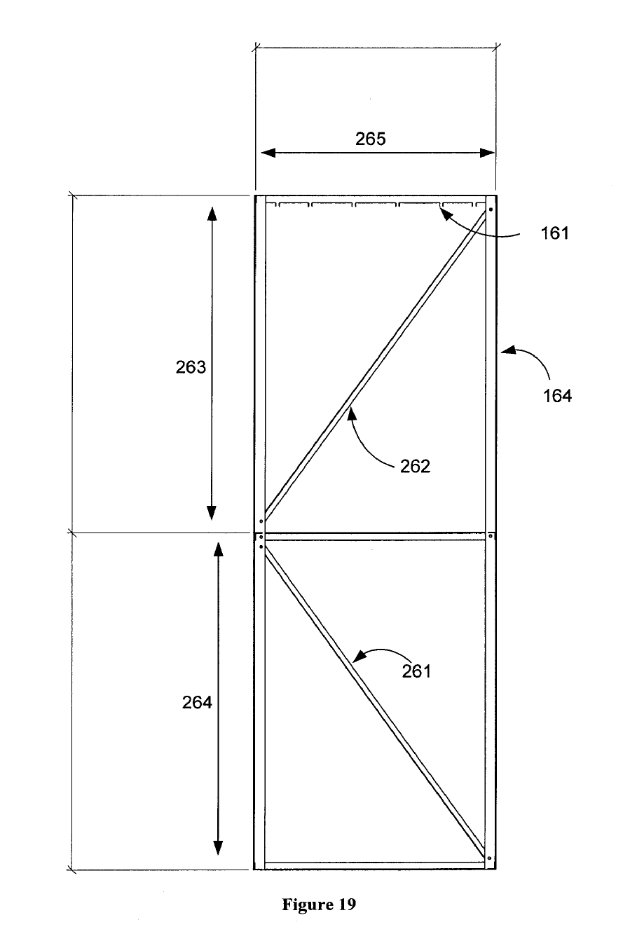

Multiple views of an embodiment of a BioPod™ are shown in FIG. 15, FIG. 16, FIG. 17, FIG. 18, and FIG. 19. FIG. 15 shows an overview of a pod. The pod can have tracks, rails, or channels for racking a plurality of blades. The tracks, rails, or channels (161) can be placed along a top side of the pod. Additionally, tracks, rails, or channels (240) can be placed along a bottom side of the pod.

FIG. 16 shows a top view of the pod. The pod can have a rack (164), also called a frame herein, that encloses a plurality of blades (31) that are secured by tracks or railing (161). Additional stability can be given to the frame using cross braces (163). Tanks and pumps can be located within the backplane portion of the pod (165). Dimensions of the pod can be any dimension known to those skilled in the art, however, as an example, the referenced dimensions of FIG. 16 can be as follows: 173—11 feet; 179—21 feet, 178—4 feet, 174—5.5 feet, 177—5.5 feet, 175—5 feet, 176—5 feet, 166—1.75 feet, 172—1.75 feet, 167—1.5 feet, 168—1.5 feet, 169—1.5 feet, 170—1.5 feet, and 171—1.5 feet.

FIG. 17 shows a front-view of the pod, facing the backplane. The front-view shows a rack or frame (164) that can support pumps, tanks and other photobioreactor components in an area inside the backplane (165). The frame can be reinforced by diagonal bracing (198). Dimensions of the pod can be any dimension known to those skilled in the art, however, as an example, the referenced dimensions of FIG. 17 can be as follows: 190—5.5 feet, 191, 5.5 feet, 192, 6 feet, 193—5.5 feet, 194—5.5 feet, 195—5.5 feet, 196—9.5 feet, and 197—11 feet.

FIG. 18 shows a side-view of the pod. The rack or frame of the pod (164) can support pumps, tanks, and other photobioreactor components in an area inside the backplane (165). The frame can be reinforced by diagonal bracing (237). The blade having a plurality of horizontal tubes (238) connected by pipe slice connections or elbow joints (239) is also depicted. The arrangement of tubes can be serpentine, winding, or zig-zag. The tubes can be supported by railing. The top railing (162) and bottom railing (240) can have rollers that facilitate entry and exit of a blade to and from the backplane. Dimensions of the pod can be any dimension known to those skilled in the art, however, as an example, the referenced dimensions of FIG. 18 can be as follows: 214—32 feet, 222—25 feet, 212—16 feet, 215—16 feet, 221—21 feet, 211—10 feet, 213—10 feet, 216—10 feet, 217—5.5 feet, 218—5 feet, 219—5 feet, 220—5.5 feet, 223—4 feet, 224—9.5 feet, 225—5.5 feet, 226—5.5 feet, 227—5.5 feet, 228—6 feet, 236—3 feet, 235—4 feet, 234—4 feet, 233—4 feet, 232—4 feet, and 231—2 feet.

FIG. 19 shows a back-view of the pod. The back-view shows a frame (164) and diagonal bracing (262, 261) for reinforcing the frame. In some embodiments of the invention, the bracing can be used to lock a blade within a pod. Dimensions of the pod can be any dimension known to those skilled in the art, however, as an example, the referenced dimensions of FIG. 19 can be as follows: 264—16 feet, 263—16 feet, and 265—11 feet.

Additional views of rack components and illustrations of welding and bolting between rack components are included in the Appendix.

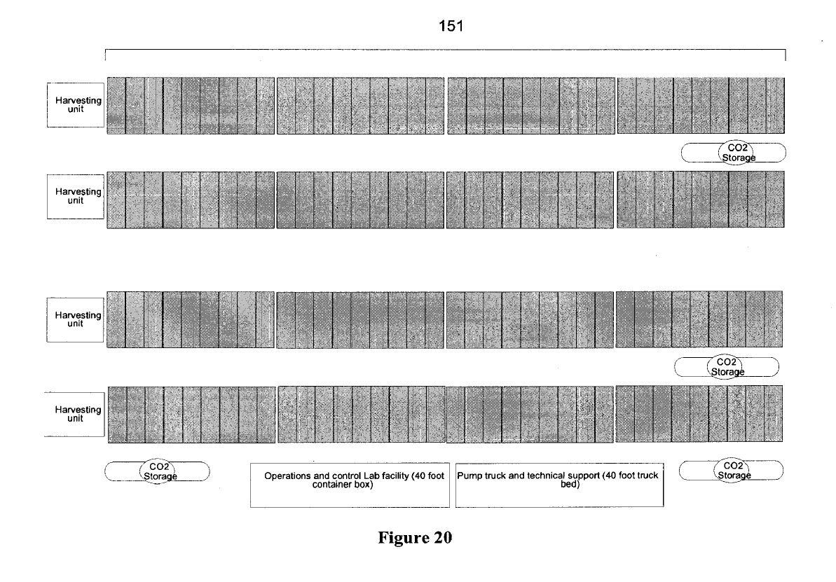

FIG. 20 shows a schematic of a photobioreactor system having a plurality of sixteen hives (151), four harvesting units, four carbon dioxide storage units, an operations and lab facility, and a pump truck. The photobioreactors can occupy a space that is approximately 250 feet by 420 feet, for a total of about 105,000 square feet. An additional 25,000 square feet can be used for support and operational equipment. The total land use can be approximately 3.2 acres.

In preferable embodiments of the invention, the pods, backplanes, and/or blades are aligned in an orthogonal manner, such that a blade can enter or exit a pod at a ninety degree angle to a row of pods that form a cluster or a hive. Alternatively, the pods, backplanes, and/or blades can be angled relative to other pods so to facilitate entry and exit of a blade. For example, angling the backplanes by 20 degrees can allow for blades to be inserted at an angle that is not perpendicular to a row of pods that form a cluster or a hive. The advantage provided by angling the blades can be a similar to the advantages of a parking lot with angled parking spots. The angling can all be in the same direction. The photobioreactors systems can be spaced about 35 feet apart to allow for entry and exit of a blade, or the spacing can accommodate the terrain of the site. In the case that the blades have an angled entry to a row of pod that form a cluster or hive, the spacing between rows of pods can be reduced.

The photobioreactor system shown in FIG. 20 can have a total of 48 reactors, having 144 pods and 864 blades. The modules of the system can include a BioBlade™, a BioPlane™, a BioPod™, a BioBloc™, and a BioHive™. The total volume of growth medium that can be contained within the blade is about 650,000 gallons or 2.5 million liters. An additional 350,000 gallons can be contained within tanks and the flow control systems in the backplanes, for a total of about 1 million gallons. The system depicted in FIG. 20 can have a capacity of greater than 1 million gallons per 100,000 square feet. The volume of growth medium contained with a given area can be increased by the vertical stacking or height of tubes within a blade, by increased density of blades, or by other means known to those skilled in the art.

The harvesting unit can be used for separation of biomass from a growth medium. The harvesting unit can separate the photoautotrophic organism from the growth medium by any methods known to those skilled in the art. Additionally, the harvesting unit can separate a biomass product other than the photoautotrophic organism from the growth medium and the photoautotrophic organism. For example, the harvesting unit can recover a biofuel, such as ethanol, butanol, or oil contained within the photobioreactor system. The harvesting unit can include a centrifuge, a distillation unit, a flash unit, a vacuum, a settling tank, or any other separation devices known to those skilled in the art.

The carbon dioxide storage units can be used to store excess carbon dioxide. Storage of carbon dioxide can better enable delivery of an appropriate amount of carbon dioxide to the photoautotrophic organisms without wasting excess carbon dioxide supply that can be produced by an industrial plant. Such an appropriate amount can be an amount that is related to the capacity of the photoautotrophic organisms to consume carbon dioxide.

The photobioreactor systems described herein, for example the system depicted in FIG. 20, can be solar powered. Sunlight can be used to generate electricity needed to power the electronics and mechanical hardware, such as pumps. Solar panels can be placed anywhere in the site, or a solar panel can be placed in relation to a given module, for example a pod, a cluster, or a hive. Alternatively, solar power can be generated offsite and directed to a photobioreactor system using transmission lines.

The photoautotrophic organisms for growth within the photobioreactor systems described herein can be any photoautotrophic organism known to those skilled in the art. A photoautotrophic organism can be any type of algae, such as spirulina or chlorella.

Example—Assembly

A photobioreactor system site is selected based on availability of resources, such as land, light, carbon dioxide, and other nutrients. Additionally, the location and environmental conditions of a site is used to determine the sites desirability. Once the site is selected, a photobioreactor system is designed based on desired system capabilities and available resources, such as capacity for carbon sequestration, and appropriate amounts of materials for the construction of the photobioreactor system are transported to the site. Specifically, the materials include tubes for constructing blades and rails for constructing the structures to support the tubes and photobioreactor components of a backplane.

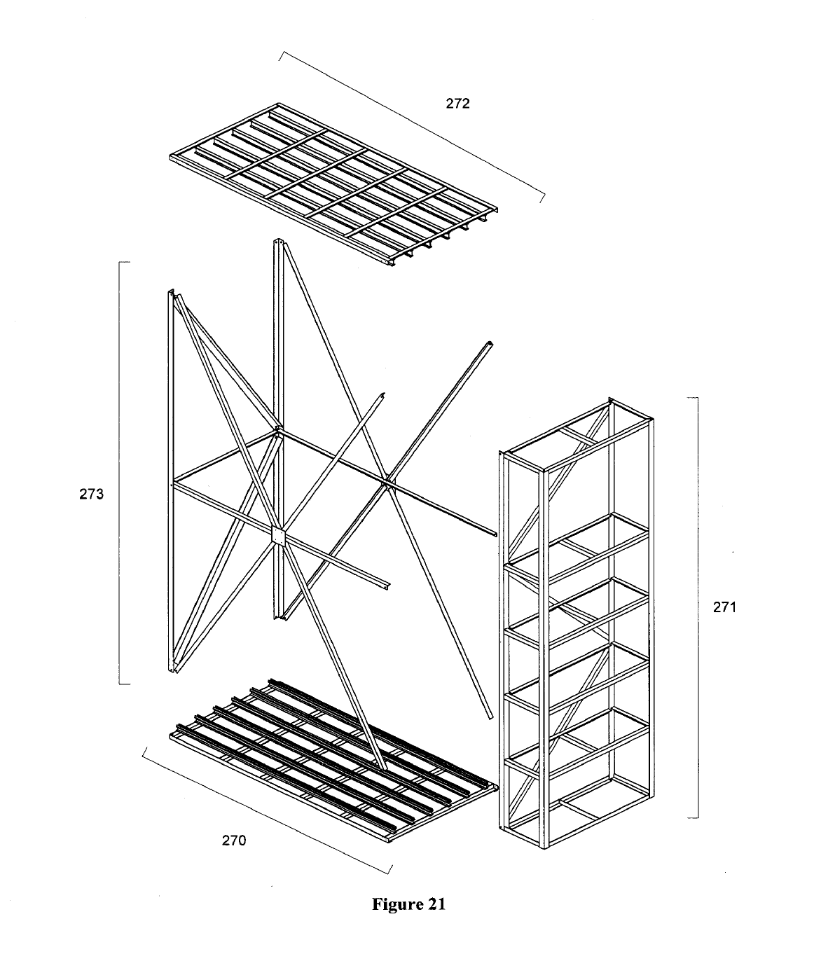

The materials include components that are easily assembled at the site and are designed for low-cost shipping. An exploded view of a rack for a pod is shown in FIG. 21. The pod can have a shop-welded tank frame (271) that can form part of the backplane, a shop-welded top frame (272) that can form the top of the pod, a shop-welded base frame that can form the bottom of the pod, and a field-assembled frame (273) that encases that pods and also couples to the tank frame (271), top frame (272), and base frame (270).

The components of the photobioreactor system and assembled and integrated with a carbon dioxide supply.

Example—Operation

A photobioreactor system having multiple hives, which include clusters, pods, and blades as described herein, and multiple harvesting units is utilized the growth of a photoautotrophic organism for carbon sequestration and production of a biomass product. A particular photoautotrophic organism is selected based on a desired target process. Potential target processes include production of biomass for combustion, carbon sequestration, production of astaxanthin, or production of a biofuel.

The photobioreactor system is filled with an appropriate growth medium. The growth medium can include water, salts, minerals, and trace metals. The growth medium can be sparged with carbon dioxide. In some cases, the growth medium is sterile. Once the growth medium is prepared a culture of the selected photoautotrophic can be introduced to the photobioreactor system. As described herein, the culture can be introduced to an inoculation tank in a pod. The culture is distributed to the multitude of inoculation tanks using a network of fluidic connections between hives, clusters, and pods. Sensors within the inoculation tanks are used to determine when the culture has reached a sufficient density and can be distributed to the blades of the pod. Once the culture is distributed to the blades of the pod, the growth of the photoautotrophic organism is maintained in a bloom state, thus increasing the efficiency of the target process. The bloom state is maintained by operating the blades under appropriate conditions by monitoring conditions like temperature, light intensity, pH, oxygen levels, salt levels, and optical density, and utilizing those parameters in an optimized control process.

During the growth of the photoautotrophic organism in the multitude of pods, specific blades may become contaminated, or be otherwise under-producing. These blades can be drained, refilled with fresh growth medium, and re-inoculated. Additionally, some blades may malfunction due to mechanical problems. These blades can be disconnected from the system and replaced with a new or repaired blade.

Once the a desired amount of biomass has been produced by the photoautotrophic organism within a blade, the growth medium, including the photoautotrophic organism, is transferred to a harvesting unit through the network of fluidic connections. The growth process within a blade can be immediately restarted once the contents of the blade have been transferred.

The harvesting unit first utilizes a settling tank to separate the photoautotrophic organism from the growth medium, and then a continuous centrifuge to provide additional separation. The photoautotrophic organism can then be compressed to harvest a desired biomass product, such as oil or astaxanthin. The remains of the photoautotrophic organism are then combusted to provide electrical energy.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A scalable photobioreactor comprising:
a plurality of blades, wherein each blade includes a plurality of fluidically connected tubes; and
a rack coupled to the plurality of blades.

2. A scalable photobioreactor comprising:
A plurality of blades; and
a rack coupled to the plurality of blades, wherein the blades are configured to slide into the rack.

3. A scalable photobioreactor comprising:
A plurality of blades; and
a backplane configured to
(a) monitor conditions in the plurality of blades;
(b) determine a plurality of desired operating setting to optimize a growth condition; and
(c) adjust operating conditions in the plurality of blades.
